(19) United States
(12) Patent Application Publication
(10) Pub. No.: US 2011/0015118 A1
(43) Pub. Date: Jan. 20, 2011

(54) C-Terminal Ifapsoriasin Fragments as Antimicrobial Peptides, the Production Thereof and Use Thereof

(75) Inventors: Jens-Michael Schroeder, Blumenthal (DE); Britta Hansmann, Kiel (DE); Zihong Wu, Kronshagen (DE)

Correspondence Address: DIEDERIKS & WHITELAW, PLC, 13885 HEDGEWOOD DR., SUITE 317, WOODBRIDGE, VA, 22193, US

(73) Assignee: UNIVERSITAETSKLINIKUM SCHLESWIG-HOLSTEIN, Kiel (DE)

(21) Appl. No.: 12/746,862

(22) PCT Filed: Nov. 28, 2008
(86) PCT No.: PCT/DE2008/001984

§ 371 Date: Sep. 15, 2010

(30) Foreign Application Priority Data
Dec. 12, 2007 (DE) 10 2007 059 891.4

(51) Int. Cl. A61K 38/17; C07K 14/47; A61K 8/64; A61P 31/04; A61P 1/00; A61P 11/00

(52) U.S. Cl. 514/2.8; 530/350; 530/324; 514/18.8; 514/21.3; 514/21.2

(57) ABSTRACT

The present invention relates to novel C-terminal ifapsoriasin fragments as antimicrobially acting peptides and their production and use as pharmaceuticals.

The invention relates to novel, antimicrobially acting peptides and the production thereof and use thereof as pharmaceuticals.

The body surface of multicellular animals represents an important boundary surface toward the environment and provides a first line of defense against penetrating microorganisms. In particular antimicrobially effective peptides that are present in large numbers in the epidermis and above all in the callus there above take part in the defense system. They control the microbial growth in the first hours after a superficial injury and during the healing of the wound. They can be found in particular in the case of some skin diseases.

Six classes of antimicrobial peptides have been discovered so far in the human skin, the 13-defensins, RNase-7, psoriasin (S100-A7), cathelicidin hCAP18, dermcidin, and lysocyme. RNase 7, lysocyme, and dermcidin are formed constitutively in healthy skin, while the β-defensin and the cathelicidin are only induced by inflammatory stimuli in human keratino-cytes and thus in the first place act as a reaction to injuries.

Defensins are small cationic and amphipatic peptides having a molecular weight of 3 to 5kDa, they have an antibacterial and an antimycotic effect. While the α-defensins HNP1-4 are for example formed in human neutrophile granulocytes that are enriched in inflamed tissue, the β-defensins hBD-2 and hBD-3 that are produced by epithelial cells are to be found in the inflamed skin and other inflamed body surfaces.

RNase 7 is a small cationic protein that is produced constitutively by epithelial cells in particular in the skin and made available. It shows a broad antimicrobial spectrum with a particularly high efficiency against Gram positive enterobacteria such as enterococci.

Psoriasin likewise is a small protein that is produced by skin keratinocytes. It is an antibiotic protein specifically effective against the enterobacterium Escherichia coli and detectable on the skin surface in antibiotically effective concentrations.

Dermcidin is a further antibiotic peptide that is specifically produced by cells of the perspiratory glands and is effective against staphylococci and Escherichia coli. It can be detected in sweat where it acts as a protective factor against infections with staphylococci.

Peptides are generally antimicrobial endogenous, gene-coded peptides with a special importance for the early phase of defense against microbial pathogens, including infection prophylaxis. Some are permanently present as antimicrobially active peptides; further ones, for example the peptide LL-37, are not formed until after the separation of larger, per se antibiotically inactive proteins, and again others are newly synthesized locally. Therefore they can be detected within minutes or hours, according to their type, after first contact with the pathogen.

However the known antimicrobial peptides are not effective against all microbial pathogens in the same way. Dermcidin for example is not effective in the case of infections with Pseudomonas aeruginosa, an important cause for skin infections in particular in the case of burns and for lung infections, in particular in the case of mucoviscidose.

For the fight against pathogenic microorganisms, also antibiotics are used for prevention or cure, that is to say substances of microbial origin that inhibit the growth of other microorganisms or even kill them off. In contrast to the antimicrobial peptides mentioned above, as a rule antibiotics have a selective effect. Many microorganisms exhibit a natural insensitivity against an antibiotic, however they can also develop this so-called antibiotics resistance during the course of the growth in the presence of antibiotics.

As a result of mutation and selection processes and also by forming resistances, there is an increasing occurrence of problems with microbial pathogens in the day-to-day clinical work and also during the production of pharmaceuticals and cosmetics that cannot be fought effectively or not at all.

Against this background there is a constant demand for new antimicrobially acting agents that can be used for prevention or for cure.

The present invention is thus based on the objects of providing antimicrobially acting peptides that can be used as pharmaceuticals that can be accessed well and have a biological and therapeutic activity of a natural substance, and of showing a way to the production thereof.

According to the invention, this object is solved by antimicrobially acting peptides having the features listed in Claims 1 to 7.

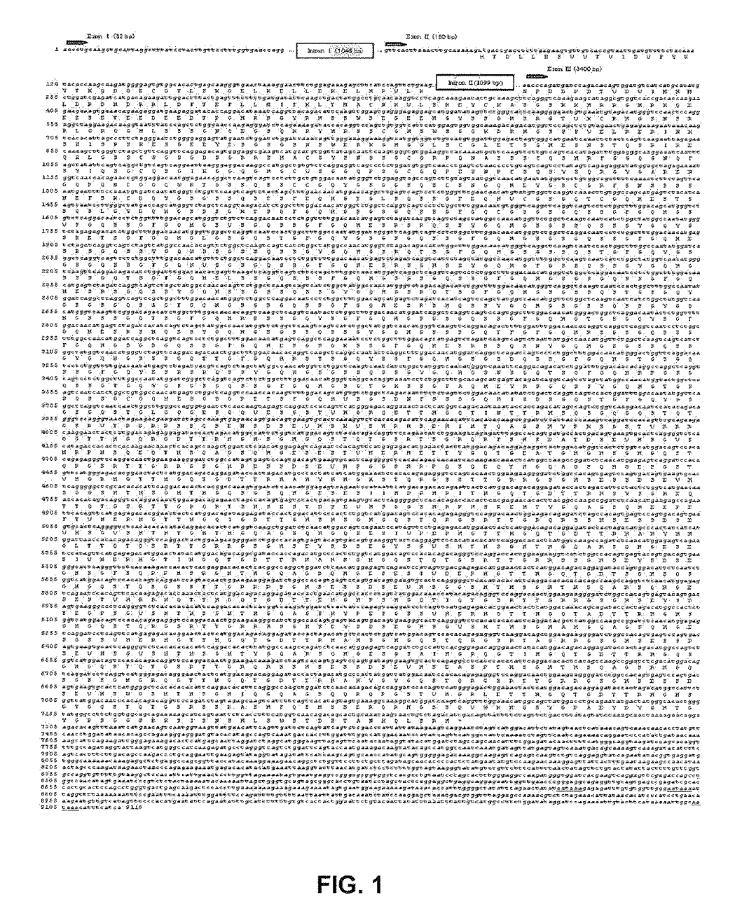

The invention is illustrated in more detail in FIGS. 1 to 5, in which

FIG. 1 shows a nucleotide and amino acid sequence of the human ifapsoriasin cDNA,

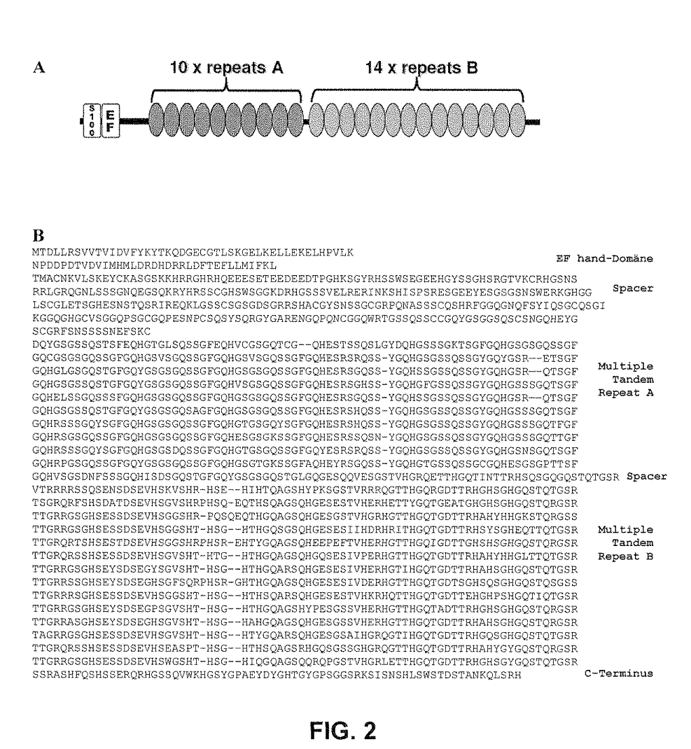

FIG. 2 shows a schematic structural representation of the human ifapsoriasin protein,

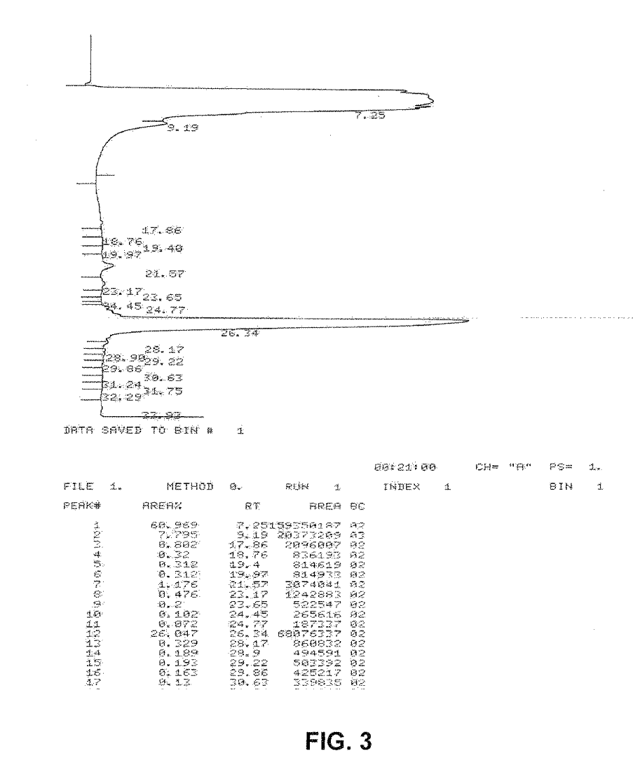

FIG. 3 shows the isolation of the IFPS₂₂₄₄₋₂₃₉₁ SUMO fusion protein by means of reverse phase HPLC,

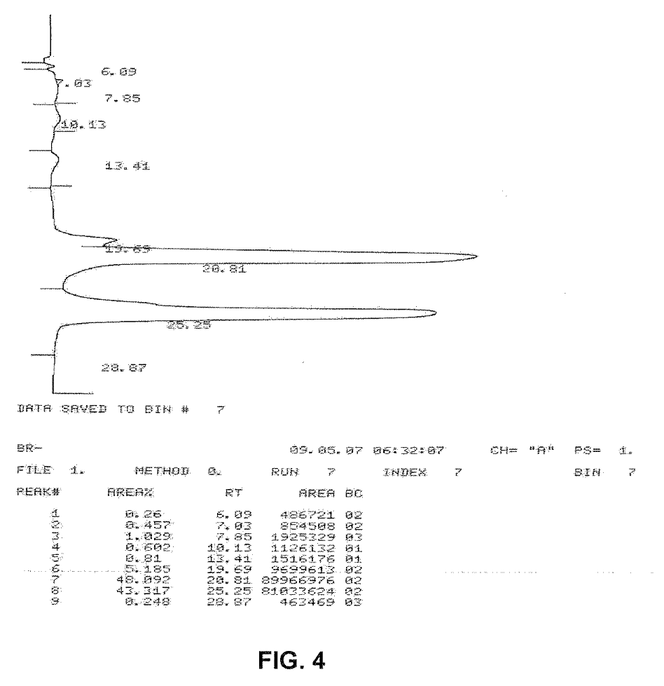

FIG. 4 shows the reverse-phase HPLC of the IFPS₂₂₄₄₋₂₃₉₁-SUMO fusion protein digestion and isolation of IFPS₂₂₄₄₋₂₃₉₁,

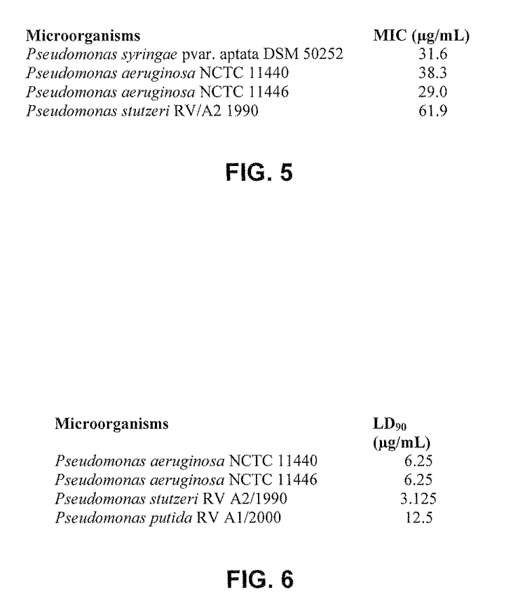

FIG. 5 shows the MIC (minimum inhibition zone concentration) for the IFPS peptide 2 (SEQ ID:NO 2), and

FIG. 6 shows LD90 (lethal dose 90, concentration at which 90% of the microorganisms are killed off) for the IFPS peptide 2 (SEQ ID: NO 2).

For the ifapsoriasin described above (below: IFPS) the coding nucleic acid sequence (cDNA) shown in FIG. 1 was found. The complete nucleotide sequence of the human ifapsoriasin cDNA (9117 bp) was confirmed by cloning and sequencing. The derived amino acid sequence of the open reading frame was illustrated directly below the coding bases. The nucleotides that have been shown in bold and underlined specify the canonic poly(A) signal sequence. The IFPS gene consists of three exons and two introns. The exon/intron boundaries were determined using the BLAST search program. The cDNA sequence data are available from the GenBank at the “National Center for Biotechnology Information” (NCBI) under the accession number AY827490. The analysis of the coding nucleotide sequence localized the gene of the new protein IFPS on the chromosome 1q21. IFPS has a very restricted expression pattern since it is formed only in human skin and vaginal mucous membrane.

The IFPS gene codes a protein with 2391 amino acids that consists of a so-called S100 part, a so-called EF-hand part, a spacer, 10 repeat domains A, 14 repeat domains B and a C-terminus (FIG. 2).

The 248kDa IFPS polyprotein is processed proteolytically in the skin to form different peptides. Using two different antimicrobial assays the inventors could demonstrate that C-terminal peptide fragments are toxic for water bugs as a function of the dose and also in a selective manner, in particular Pseudomonas aeruginosa, Pseudomonas stutzeri and Pseudomonas syringae.

The present invention also provides a production method for the inventive peptides and the use of the inventive peptides as pharmaceuticals for different therapeutic and diagnostic indications. For this purpose, the peptides can be used as high-purity substances or—if adequate for the application—within a partly purified peptide mixture or as a mixture of several inventive peptides or also their gene probes.

Against this background, the invention further relates to nucleic acid molecules with a sequence section coding for an inventive peptide, an expression vector with such a nucleic acid molecule and, if desired, control sequences in particular for replication, transcription and/or translation, and a host cell that is transfected or transformed using the expression vector. To this end, different expression vectors are routinely available for secretory or direct cytoplasmatic expression.

After the amino acid sequence of the inventive peptides is known, a corresponding nucleic acid sequence can be derived using the genetic code, it being possible to use optimized codons for different hosts (bacteria, yeast, mammalian cells, plants). However the choice of codon resulting from. FIG. 1 is preferred.

When peptides are synthesized as fusion proteins, the production and purification of the inventive peptides can be made easier. For amino acid sections or domains of known protein coding sequences, nucleic acids coding for the inventive peptides are fused on so that a continuous protein can be produced during the expression. Examples for such fused-on amino acid sections are the histidine “tags” which can be used to purify expressed fusion proteins on nickel chelate columns, or antigene determinants that permit the peptides to be purified on suitable antibody affinity columns.

In an exemplary embodiment it is preferred if the peptide is joined with a further peptide or protein to form a fusion protein, a SUMO protein, a histidine “tag”, a proteolytic cleavage “tag” and the IFPS peptide being used, preferably in the order specified.

Since IFPS is naturally more often found in humid skin and mucous membrane areas, the inventive peptides are particularly well suited for treating diseases that occur when organs are populated by water bugs, in particular by Pseudomonas aeruginosa.

Due to the biological effect and the natural occurrence of the inventive IFPS peptides it has been shown that the inventive preparations can be employed as agents for the therapy of infectious diseases of many epithelial organs and for the infection prophylaxis on boundary surfaces, in particular of the skin, the nasopharyngeal area, the eyes, the respiratory tract, the gastrointestinal tract, and the urogenital tract.

The inventive peptides can also be used for treating systemic diseases in the case of a lack of these peptides for treating diseases of the human organism, in particular when the respiratory tract, the gastrointestinal tract, and the urogenital tract are involved.

In a further embodiment of the invention it is also possible to use the inventive peptides for treating chronic diseases, partly accompanied by the diseases already mentioned, in that they are used in a suitable form for the treatment.

The inventive peptides can further be used for treating diseases in the acute stadium.

Against this background the invention further relates to a pharmaceutical or cosmetic composition that contains an inventive peptide in an antimicrobially effective amount as effective ingredient, preferably in the range of 1-50 μg/mL.

In a further embodiment the inventive peptides can be used to coat materials, preferably catheters, contact lenses, or orthopedic implants.

Due to the particularly high effectiveness of the inventive peptides against pathogenic soil germs, the inventive peptides can also be used to protect plants, preferably by producing transgenic plants by cloning the nucleic acids that code for the inventive peptides.

The inventive peptides are also suitable to be used for infection prophylaxis and for treating infections in animals, preferably farm animals and domestic animals.

The inventive peptides can be used for diagnosing diseases already mentioned above in that for example antibodies against one or more of the inventive peptides or of their derivatives or its fragments are produced and the concentration of one or more inventive peptides in body fluids or tissues is measured using immunological methods.

The IFPS peptides can also be used as pharmaceuticals. They contain one or more of the inventive new IFPS peptides or a physiologically acceptable salt of these peptides. The form and composition of the pharmaceuticals that contain one or more of the new IFPS peptides is governed by the way they are administered. The pharmaceuticals containing one or more of the new IFPS peptides can be administered topically, parenterally, intranasally, orally, or by means of inhalation. These pharmaceuticals containing one or more of the new IFPS peptides are preferably packaged in formulations suitable for the topical use, preferably creams, ointments, or solutions, or with an injection preparation, either as a solution or as lyophilisate for dissolving immediately before use. The pharmaceutical preparations can also contain substances that are necessary technically for filling, contribute to the solubility, stability, or sterility of the pharmaceutical or increase the efficiency of the absorption into the body.

The determination of the biological activity for the inventive new IFPS peptides is based on measurements relative to internationally accepted reference preparations for antibiotic substances. The inventive new IFPS peptides are suited in particular also for a long-term therapy in the case of infectious diseases since they have an excellent biological effectiveness and specificity against water bugs, in particular Pseudomonas aeruginosa, and as endogenous peptides on the other hand do not trigger any immune reactions even during long-term treatment.

To determine their activity, the inventive IFPS peptides were tested for their antimicrobial effect. In the radial diffusion assay the activities specified in FIG. 5 could be measured against different bacterial strains.

The isolation of the IFPS cDNA, its presence in different tissues, the production of a recombinant IFPS peptide, and the proof of its antimicrobial activity are described in the following examples.

1) Isolation of the IFPS cDNA and Determining the Genome Sequence

In in silico analyses of the human genome with the aid of the “Ensembl” server, a sequence having a length of 82 amino acids consisting of the conserved “EF-hand” domains of the profilaggrin was used to look for putatively new members of the “S100 Fused Type Protein” (SFTP) family. Among others a sequence was found between the known genes for profilaggrin (FLG) and cornulin (CRNN or Clorf10), after which a hypothetical cDNA sequence was established by exon analysis of the surrounding genome region by means of the program “FuzzyFinder”. The region that was examined contained two putative exons. The total cDNA sequence was determined from keratinocyte cDNA. To this end the entire RNA was isolated from human foreskin keratinocytes using TRIzol® according to protocol. After DNAse digestion 3 μg of the total RNA was transcribed into cDNA using the SMART RACE cDNA Amplification Kit (Clontech, Heidelberg, Germany). By means of 5′ and 3′ RACE (“Rapid Amplification of cDNA Ends”) the terminal areas of the cDNA were determined; the exon3 having a size of almost 9kb of the IFPS was amplified via the “Short-Range Overlapping PCR” method and then sequenced. The complete cDNA sequence is available from the “National Center for Biotechnology Information” (NCBI) using the GenBank accession number AY827490. The IFPS cDNA sequence has an overall length of 9117 bp, has an open reading frame of 7176 bp and a polyadenylation signal (AATAAA) nine nucleotides 5′ before the start of the polyadenylation. It is structured similar to the cDNA sequence of all previously known “SFTP” having a total of two introns (1048 and 1089 bp) and three exons (52, 160, and 8907 bp) of which only the last two carry the protein-coding sequence. The derived protein sequence consists of 2391 amino acids and has an N-terminal S100 and EF-hand domain, then a spacer region followed by two different multiple tandem repeats regions (10×A and 14×B) both of which have a length of 75-77 amino acids but differ as to their sequence and are separated by a short spacer, and finally the C-terminus.

### 2) Proof of IFPS in Different Tissue Samples

To determine the IFPS mRNA expression, an RT-PCR was carried out on cDNA samples of different human tissues and cells using intron spanning primers. In the process the IFPS mRNA could be detected only in the skin and in cultivated foreskin keratinocytes. No mRNA could be found in all of the tissues that were examined like spleen, thymus, small intestine, bone marrow, tonsils, stomach, liver, larynx, pharynx, colon, polyps, lung, salivary gland, kidney, uterus, lymph nodes, neutrophiles, bronchial, and tracheal epithelial cells. In cultivated keratinocytes, the IFPS mRNA is increased strongly by adding Ca²⁺ to the medium, so that it can be assumed that IFPS, like profilaggrin and involucrin also, could be used as marker for keratinocyte differentiation.

### 3) Construction, Expression, and Purification of a Recombinant IFPS Peptide

A fragment of the IFPS that includes the last B repeat domain and the complete C-terminus (148 amino acids) were produced recombinantly in the SUMO system (Invitrogen, Karlsruhe, Germany; LifeSensors, Malvern, UK).

To this end the C-terminal fragment was amplified by means of PCR of keratinocyte cDNA with a Pfu-polymerase. The amplified PCR fragment was then additionally incubated with a Taq-polymerase to generate desoxyadenosin overhangs at the 3′ ends of the PCR product, using which the fragment was ligated into the pET SUMO vector. The vector was transformed into chemocompetent E. coil TOP10 cells, and positive clones were identified via the antibiotics resistence (here kanamycin) and colony PCR. The vector was isolated and sequenced from overnight cultures in LB medium and a corresponding antibiotic (50 μg/mL) and transformed at the correct nucleotide sequence in E. coli BL21(DE3)pLysS cells that are capable of protein expression. Positive clones were identified as before.

The expression of the fusion protein in BL21(DE3)pLysS took place at 37° C. and 200 rpm in LB liquid medium with 14mM glucose with the addition of 34 μg/mL chloramphenicol and 50 μg/mL kanamycin. The culture was induced at an optical density (OD) of 0.4-0.6 at a wavelength of 600 nm with IPTG and incubated for another three hours. Then the cells were pelleted, suspended in 1 mL lysis equilibration buffer (LEW) for each 100mL culture and stored at −80° C.

Breaking the cells was carried out by thrice thawing and freezing at 30° C. and −80° C. (“freeze and thaw” method) and then ultrasound treatment, the volume being between 4 and 5mL for each 50mL vial. The ultrasound treatment was carried out on ice for 5 mins, in each case in cycles with 30 sec ultrasound (60/40 interval) and 15 sec break. The cell residues were centrifuged off at 4° C. and 12000 g for 45 mins and the excess was sterile filtered.

Further purification took place via a nickel chelate column (Protino® Ni-IDA 1000 packed columns, Macherey-Nagel, Düren, Germany). Here the fusion protein was purified according to protocol by interactions between the histidine of the additionally expressed tag and the nickel ions bound to the column matrix.

To rule out as far as possible any contamination by bacterial protein, the fusion protein was further purified via a reversed phase (RP) HPLC with an RP8 column and precolumn (SP 250/10 or SP 50/10 NUCLEOSIL 300-7 C8, Macherey-Nagel, Düren, Germany) across a linear gradient (FIG. 3). Here buffer A was water with 0.1% trifluoroacetic acid (TFA), buffer B acetonitrile with 0.1% TFA and the gradient as follows: 0-5min 90% A/10% B, up to 30 min linear to 40% A/60% B, in 5 min linear to 100% B; the flow rate was 3mL/min and detection took place at 215 nm.

The fraction obtained with a retention time of 26.34 min was collected and analyzed by mass spectrometry using the ESI method to check the size of expressed protein. The protein of this fraction (IFPS₂₂₄₄₋₂₃₉₁ SUMO fusion protein) had the correct size of 29141.3Da; the concentration was determined photometrically using the absorption coefficient, and the fraction was freeze-dried.

For digestion, 100 μg fusion protein was in each case taken up in 500 μL 1×PBS (Phosphate Buffered Saline) and incubated with 2.5U SUMO Protease1 (LifeSensors, Malvern, UK) for at least 3 h at 30° C. Separating the fusion tag and the possibly undigested fusion protein took place via RP-HPLC using a RP4 column (Bakerbond C4 5 u 300 A, 250×4.60 mm micron, Phenomenex, Aschaffenburg, Germany) (FIG. 4). Here buffer A was water with 0.1% trifluoroacetic acid (TFA), buffer B acetonitrile with 0.1% TFA and the gradient as follows: starting concentration 90% A/10% B, up to 10 min linear to 60% A/40% B, in 15 min linear to 30% A/70% B, in further 10 min to 100% B; the flow rate was 0.5mL/min and detection took place at 215 nm.

The fraction obtained with a retention time of 20.81 min (corresponding to 55% acetonitrile) was collected and analyzed by mass spectrometry using the ESI method to check the size of expressed protein (IFPS₂₂₄₄₋₂₃₉₁). The protein of this fraction had the correct size of 15874.5Da; the concentration was determined photometrically using the absorption coefficient and the fraction was freeze-dried for further use.

### 4) Proof of Antimicrobial Activity in the Radial Diffusion Inhibition Assay

In the radial diffusion inhibition assay, the antimicrobial activity of peptides, for example a C-terminal IFPS fragment, is examined against microorganisms suspended in agarose, here for example Pseudomonas aeruginosa. For this purpose, proteins to be examined as mentioned in example 3 are purified and lyophilized and suspended in 0.01% acetic acid.

To test the antimicrobial activity the following procedure is carried out: 4×10⁶cfu (colony forming units) from a logarithmically growing culture in TSB (Tryptic Soy Broth) medium (Sigma) are added to a 42° C. warm agarose solution (1% TSB, 10mM sodium phosphate buffer pH 7.4, 1% agarose, 0.02% Tween 20). After cooling the agarose solution in plates, cavities having a diameter of 3 mm are stamped into the solid agarose under sterile conditions.

5 μL each of a peptide solution to be tested are added to the cavities and incubated over night at 37° C. The solvent 0.01% acetic acid is the negative control; human lysocyme (500ng/mL, Sigma) is the positive control.

The following day another 42° C. warm nutrient-rich agarose solution (1% agarose, 4% peptone, 0.5% glucose, 1% sodium chloride, 0.5% potassium hydrogen phosphate, pH 7.2) is poured into the plates and incubated for another three to four hours in the incubator. Inhibition zones that have developed are measured to evaluate the antimicrobial activity.

5) Proof of the Antimicrobial Activity against Pseudomonas aeruginosa in the Micro Dilution Inhibition Test.

The antimicrobial activity of the IFPS fragments is additionally determined in a liquid-culture test system. For this purpose the different Pseudomonas aeruginosa strains are cultivated in BHI (Brain Heart Infusion) medium, diluted in 10mM sodium phosphate buffer (pH 7.3) with 1% TBS and 90 μL of this is incubated with 10 μL of the peptide solution (in different concentrations) for 2 h at 37° C.

After the incubation time, the charges are diluted 1:10 and 1:100 in. 10mM sodium phosphate buffer (pH 7.3) with 1% TBS. Of these dilutions, 100 μL each are plated in three parallel charges on BHI agar.

After incubating the plates for 24 h at 37° C. the colony forming units (cfu) are counted. As a control, one charge each with only 10mM sodium phosphate buffer is plated directly before and after the two-hour incubation.

6) Antimicrobial Activity of C-terminal IFPS Peptides against Pseudomonas ssp.

The antimicrobial activity of the IFPS peptide was examined against different microorganisms using both the radial diffusion and the micro dilution inhibition test.

The minimum inhibition zone concentration (MIC) designates that minimum peptide concentration that is required to completely inhibit the growth of some Pseudomonas ssp after the incubation time (cf. Example 5). For the C-terminal IFPS peptide 2 the minimum inhibition zone concentration specified as an example in FIG. 5 was found, the concentration referring to the 5 μL volume filled into the cavity.

The lethal dose at which 90% of the microorganisms are killed off (LD₉₀) can be determined from the reduced growth of the cfu in the micro dilution inhibition test. The LD₉₀ values for some Pseudomonas ssp are specified in FIG. 6.

1. A use of a peptide having the sequence shown in SEQ ID:NO 1, SEQ ID:NO 2, or SEQ ID:NO 3 for the production of an antimicrobially acting agent.

2. The use according to claim 1 for the production of a pharmaceutical for treating infectious diseases of the human or animal epithelial tissue.

3. The use according to claim 1 for treating infectious diseases of the human organism involving the skin, the gastrointestinal tract, the respiratory tract, or the urogenital tract.

4. The use according to claim 1 for treating infections with bacteria of the species Pseudomonas for the production of a pharmaceutical for substitution therapy.

5. The use according to claim 1 for treating chronic infections with bacteria of the species Pseudomonas.

6. The use according to claim 4 for treating infections with Pseudomonas aeruginosa.

7. The use according to claim 1, characterized in that in SEQ ID:NO 1 position 96 (G) has been substituted with D and/or in position 98 (S) with D, G, N or Q, in position 100 (V) with G or A, in position 102 (K) with R or T, in position 108 (P) with S or N or R, in position 112 (D) with E or N or Y, in position 115 (H) with Q or E or K, in position 116 (T) with S, in position 118 (Y) with F, in position 123 (G) with R or D or A, in position 124 (S) with R or L and/or in position 125 (R) with Q or R.

8. The use according to claim 1 for coating medical instruments.

9. The use according to claim 1 for coating catheters, medical implants, or contact lenses.

10. A medical instrument, catheter, medical implant, or contact lens, characterized by a coating having a peptide with the sequence shown in SEQ ID:NO 1, SEQ ID:NO 2, or SEQ ID:NO 3

11. A cosmetic composition, characterized by a peptide having the sequence shown in SEQ ID:NO 1, SEQ ID:NO 2, or SEQ ID:NO 3.

12. The cosmetic composition according to claim 11, characterized in that the concentration of the peptide is 1 to 50 μg/mL.

13. A pharmaceutical, characterized by a peptide having the sequence shown in SEQ ID:NO 1, SEQ ID:NO 2, or SEQ ID:NO 3.

14. The pharmaceutical according to claim 13, characterized in that the concentration of the peptide is 1 to 50 μg/mL.

15. The use according to claim 2 for treating infectious diseases of the human organism involving the skin, the gastrointestinal tract, the respiratory tract, or the urogenital tract.

16. The use according to claim 2 for treating infections with bacteria of the species Pseudomonas for the production of a pharmaceutical for substitution therapy.

17. The use according to claim 3 for treating infections with bacteria of the species Pseudomonas for the production of a pharmaceutical for substitution therapy.

18. The use according to claim 2 for treating chronic infections with bacteria of the species Pseudomonas.

19. The use according to claim 5 for treating infections with Pseudomonas aeruginosa.

20. The use according to claim 2, characterized in that in SEQ ID:NO 1 position 96 (G) has been substituted with D and/or in position 98 (S) with D, G, N or Q, in position 100 (V) with G or A, in position 102 (K) with R or T, in position 108 (P) with S or N or R, in position 112 (D) with E or N or Y, in position 115 (H) with Q or E or K, in position 116 (T) with S, in position 118 (Y) with F, in position 123 (G) with R or D or A, in position 124 (S) with R or L and/or in position 125 (R) with Q or R.

21. The use according to claim 3, characterized in that in SEQ ID:NO 1 position 96 (G) has been substituted with D and/or in position 98 (S) with D, G, N or Q, in position 100 (V) with G or A, in position 102 (K) with R or T, in position 108 (P) with S or N or R, in position 112 (D) with E or N or Y, in position 115 (H) with Q or E or K, in position 116 (T) with S, in position 118 (Y) with F, in position 123 (G) with R or D or A, in position 124 (S) with R or L and/or in position 125 (R) with Q or R.

22. The use according to claim 4, characterized in that in SEQ ID:NO 1 position 96 (G) has been substituted with D and/or in position 98 (S) with D, G, N or Q, in position 100 (V) with G or A, in position 102 (K) with R or T, in position 108 (P) with S or N or R, in position 112 (D) with E or N or Y, in position 115 (H) with Q or E or K, in position 116 (T) with S, in position 118 (Y) with F, in position 123 (G) with R or D or A, in position 124 (S) with R or L and/or in position 125 (R) with Q or R.

23. The use according to claim 5, characterized in that in SEQ ID:NO 1 position 96 (G) has been substituted with D and/or in position 98 (S) with D, G, N or Q, in position 100 (V) with G or A, in position 102 (K) with R or T, in position 108 (P) with S or N or R, in position 112 (D) with E or N or Y, in position 115 (H) with Q or E or K, in position 116 (T) with S, in position 118 (Y) with F, in position 123 (G) with R or D or A, in position 124 (S) with R or L and/or in position 125 (R) with Q or R.

24. The use according to claim 6, characterized in that in SEQ ID:NO 1 position 96 (G) has been substituted with D and/or in position 98 (S) with D, G, N or Q, in position 100 (V) with G or A, in position 102 (K) with R or T, in position 108 (P) with S or N or R, in position 112 (D) with E or N or Y, in position 115 (H) with Q or E or K, in position 116 (T) with S, in position 118 (Y) with F, in position 123 (G) with R or D or A, in position 124 (S) with R or L and/or in position 125 (R) with Q or R.


SEQUENCE LISTING (3 sequences)

SEQ ID NO: 1 - protein, 148 aa, Homo sapiens
GSRTTGRRGSGHSESSDSEVHSWGSHTHSGHIQGQAGSQQRQPGSTVHGRLETTHGQTGDTTRHGHSGYGQSTQTGSRSSRASHFQSHSSERQRHGSSQVWKHGSYGPAEYDYGHTGYGPSGGSRKSISNSHLSWSTDSTANKQLSRH

SEQ ID NO: 2 - protein, 70 aa, Homo sapiens
SSRASHFQSHSSERQRHGSSQVWKHGSYGPAEYDYGHTGYGPSGGSRKSISNSHLSWSTDSTANKQLSRH

SEQ ID NO: 3 - protein, 55 aa, Homo sapiens
SSRASHFQSHSSERQRHGSSQVWKHGSYGPAEYDYGHTGYGPSGGSRKSISNSHL